(19) United States
(12) Patent Application Publication
(10) Pub. No.: US 2017/0188945 A1
(43) Pub. Date: Jul. 6, 2017

(54) MULTI FUNCTION SMART WRISTLETS

(71) Applicants: Kai Chun Lin, Fremont, CA (US); Erika Yang, Livermore, CA (US)

(72) Inventors: Kai Chun Lin, Fremont, CA (US); Erika Yang, Livermore, CA (US)

(21) Appl. No.: 15/085,531

(22) Filed: Mar. 30, 2016

Related U.S. Application Data
(60) Provisional application No. 62/272,693, filed on Dec. 30, 2015.

(51) Int. Cl. A61B 5/00; A61B 5/11; A61B 5/0205

(52) U.S. Cl. CPC A61B 5/4845; A61B 5/681; A61B 5/742; A61B 5/7475; A61B 5/0205; A61B 5/1118; A61B 5/1112; A61B 5/746; A61B 2560/0242; A61B 5/02438

(57) ABSTRACT

The embodiments disclose a new type of multi-function smart wristlets, in which a variety of electronic, optoelectronic, and thermal sensors are embedded. These sensors will collect data through various signals from both a person and the environment in order to perform many functions to help people improve their health; it also tells people of the environmental conditions in order for them to make the right decision about their outdoor activities. Also, this wristlet can be a sensor-key to open cars, house doors, garage doors, and so on. It also works as music player to play music in wireless headphones through Bluetooth function on the wristlet. In addition, at night it can also measure your steady heartbeat and blood flow rate along with breathing conditions through the detector sensors. This can monitor a person's health and sleeping quality. This function can also help to alert other family members through an app downloaded on family's phone if a condition appears in sleep or heart rate becomes irregular.

### BRIEF DESCRIPTION OF THE DRAWINGS

FIG. 1 shows a diagram of the general idea of the multi-function smart wristlet 100 where a variety of electronic sensor 110, optoelectronic sensor 120 and thermal sensor 130 are embedded. These sensors will collect data from variety of signals from the person and the environment in order to perform many functions to help people improve their health; it also tells people of the environmental conditions in order for them to make the right decision about their outdoor activities.

FIG. 2 shows a diagram of a multi-function smart wristlet 100 comprises of many functional buttons.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview

It should be noted that the descriptions that follow, for example, in terms of multi-function smart wristlet is described for illustrative purposes and the underlying system can apply to any number and multiple types sensors or buttons. In one embodiment of the present invention, a multi-function smart wristlet includes a variety of electronic, optoelectronic and thermal sensors. These sensors are embedded in the wristlet.

These sensors embedded in the multi-function smart wristlet will collect data from variety of signals from the person and the environment in order to perform many functions to help people improve their health; it also tells people of the environmental conditions in order for them to make the right decision about their outdoor activities.

This multi-function smart wristlet can also be a sensor-key to open car, house door, garage door and so on. It also works as music player to play music in wireless headphones through Bluetooth function on the wristlet.

In addition, at night it can also measure your steady heartbeat and blood flow rate along with breathing conditions. This can monitor a person's health and sleeping quality. This function can also help with alerting other family members if a condition appears in sleep or heart rate becomes irregular through an app downloaded on family's phone.

The detailed description of multi-function smart wristlet's functions is summarized below.

### DETAILED DESCRIPTION

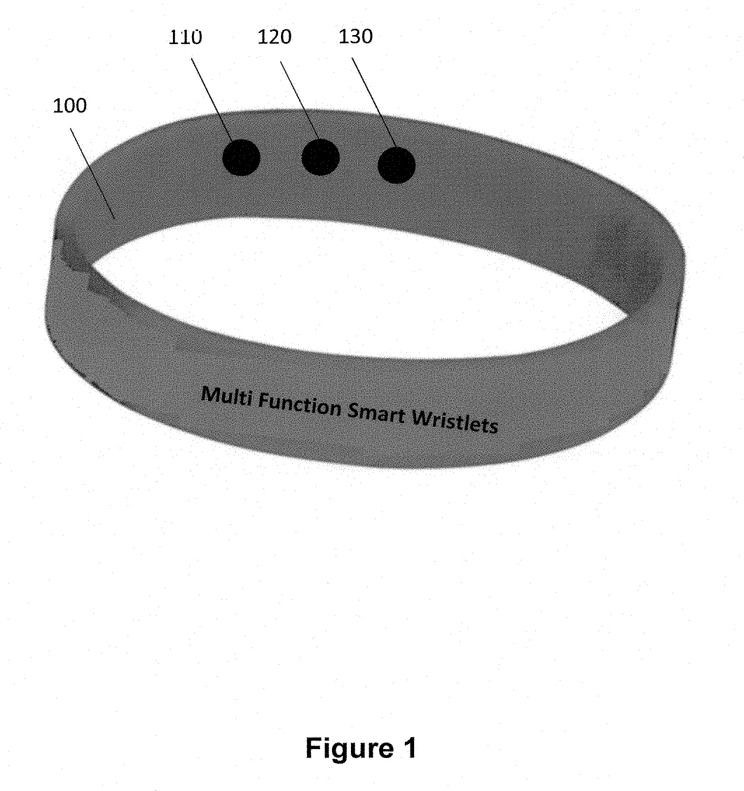

FIG. 1 shows a diagram of the general idea of the multi-function smart wristlet 100 where a variety of electronic sensor 110, optoelectronic sensor 120 and thermal sensor 130 are embedded. These sensors may electronically interconnect with each other, and they will collect data from variety of signals from the person and the environment in order to perform many functions to help people improve their health; it also tells people of the environmental conditions in order for them to make the right decision about their outdoor activities.

These different types of sensors mentioned in FIG. 1 are all embedded in the wristlet 100.

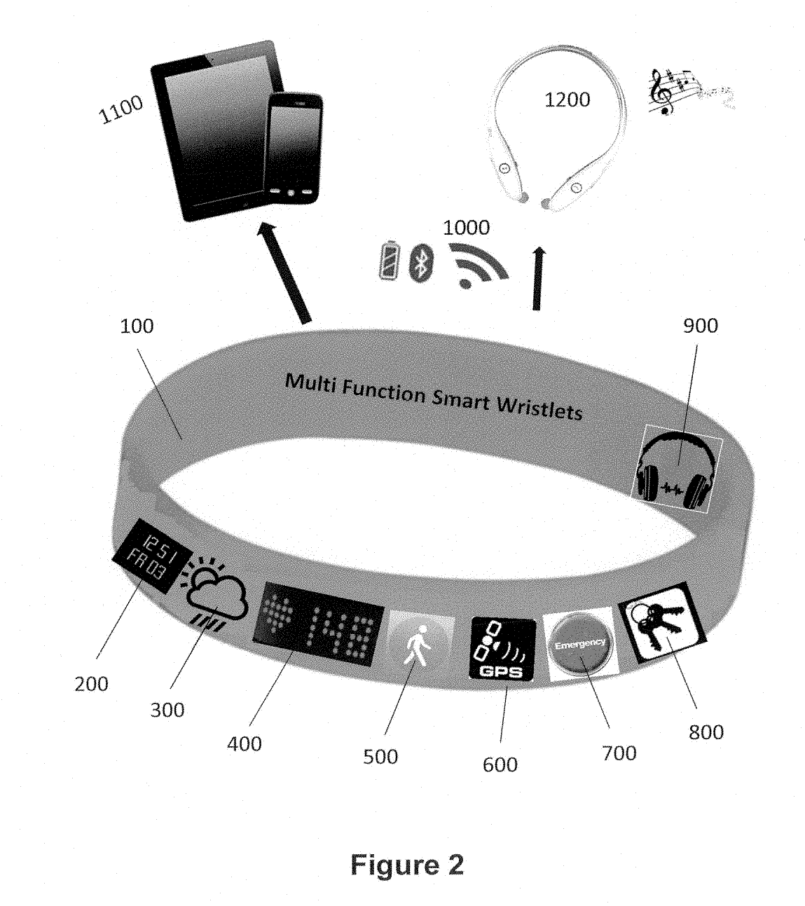

FIG. 2 shows a diagram of a multi-function smart wristlet. A multi-function smart wristlet 100 comprises of many functional buttons.

The multi-function smart wristlet, as shown in FIG. 2, is embedded with many function buttons that will be claimed below.

The multi-function smart wristlet 100 has a watch button 200, shown in FIG. 2. This button is a time function button, which can perform all digital watch functions, including but not limited to displaying date, time, setting alarms, and acting as a stopwatch. The multi-function smart wristlet 100 has an indicator of environmental conditions 300, shown in FIG. 2. It can display weather condition, temperature, UV intensity, humidity, air quality, noise intensity, electromagnetic wave intensity, blood alcohol concentration (BAC), and so on. These data will guide people's choices and improve their health condition, along with their quality of living;

The multi-function smart wristlet 100 has a health status button 400, as shown in FIG. 2. The button 400 displays the heart rate when pressed. The heart rate monitor will call emergency services and family members through wireless connection (will be mentioned later on) upon detecting palpitations. There will be an emergency button in the multi-function smart wristlet.

The health status button 400 can also be used as sleep quality monitor to measure a person's steady heartbeat and blood flow rate, along with breathing conditions through the detector sensors;

The multi-function smart wristlet 100 has a pedometer for tracking steps 500, as shown in FIG. 2. The number of steps taken can be displayed by pressing the button. The pedometer 500 not only counts steps, it will be able to connect with the heart rate monitor and the weather button to reflect the person's health condition and exercise level.

The multi-function smart wristlet 100 has a safety tracker button 600, as shown in FIG. 2. A GPS tracker for parents to ensure the safety of 1) their children 2) people with mental and/or health conditions 3) the elderly by sending their locations.

The function is not limited to these groups of people mentioned above; the safety tracker can be used for anyone as needed in general.

The multi-function smart wristlet 100 has an emergency button 700, shown in FIG. 2. The button sends an alert signal to the authorities and family members through wireless connection when pressed.

The emergency button 700 can be voice activated through the person's verbal commands.

The multi-function smart wristlet 100 has a digital key 800, shown in FIG. 2. A digital remote on wrist that can open up different locks such as garage, house door, and car etc. It is not limited to the functions mentioned above; other similar functions may also be used with the digital keys 800.

The multi-function smart wristlet 100 has a music player button 900, shown in FIG. 2. It can wirelessly, through Bluetooth 1000, hook up with any Bluetooth device 1200 and play music through them.

The music player button 900 can also be embedded with an electronic chip to perform the radio function. This button can have “we chat” function that people can talk with each other using the multi-function smart wristlet.

All data obtained from this wristlet can be uploaded onto a synced app 1100 through Bluetooth 1000.

The synced app 1100 contains recorded data that can be used for reviewing health conditions and can be accessed by authorities such as doctors, nurses, etc.

The multi-function smart wristlet 100 is not limited to these functional buttons aforementioned. It can be extended to include other functional buttons in general as needed.

Individual functions of each button can be coordinated to perform other functions not aforementioned.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1. A multi-function smart wristlet 100 shown in FIG. 1, comprising: an electronic sensor 110, an optoelectronic sensor 120, and a thermal sensor 130. However, it is not limited to these three types of sensors; other similar sensors may also be used in this wristlet. A multi-function smart wristlet 100 comprises of many functional buttons, as shown in FIG. 2.

2-19. (canceled)

20. All sensors mentioned in the claim 1 will be used to collect a person's personal health data from a variety of signals in order to perform many functions to help people improve their health.

21. These sensors mentioned in the claim 1 will collect data not only from the user, but also from the environmental data, such as the weather condition, temperature, UV intensity, humidity, air quality, noise intensity, electromagnetic wave intensity, Mood alcohol concentration (BAC), and so on. These data are used in order to perform many functions to help people improve their health.

22. The multi-function smart wristlet mentioned in claim 1 is embedded with many function buttons, as shown in FIG. 2.

23. The multi-function smart wristlet 100 mentioned in the claim 1 has a watch button 200, as shown in FIG. 2. This button is a time function button, which can perform all digital watch functions, including but not limited to displaying date, time, setting alarms, and acting as a stopwatch. The multi-function smart wristlet 100 has an indicator of environmental conditions 300, shown in FIG. 2. It can display weather condition, temperature, UV intensity, humidity, air quality, noise intensity, electromagnetic wave intensity, blood alcohol concentration (BAC), and so on. These data will guide people's choices and improve their health condition, along with their quality of living.

24. The multi-function smart wristlet 100 mentioned in the claim 1 has a health status button 400, as shown in FIG. 2. The button 400 displays the heart rate when pressed. The heart rate monitor will call emergency services and family members through wireless connection (will be mentioned later on) upon detecting palpitations. There will be an emergency button in the multi-function smart wristlet.

25. The health status button 400 mentioned in the claim 1 can also be used as sleep quality monitor to measure a person's steady heartbeat and blood flow rate, along with breathing conditions through the detector sensors.

26. The multi-function smart wristlet 100 mentioned in the claim 1 has a pedometer for tracking steps 500, as shown in FIG. 2. The number of steps taken can be displayed by pressing the button. The pedometer 500 not only counts steps, it will be able to connect with the heart rate monitor and the weather button to reflect the person's health condition and exercise level.

27. The multi-function smart wristlet 100 mentioned in the claim 1 has a safety tracker button 600, as shown in FIG. 2. A GPS tracker for parents to ensure the safety of 1) their children 2) people with mental and/or health conditions 3) the elderly by sending their locations. The function is not limited to these groups of people mentioned above; the safety tracker can be used for anyone as needed in general.

28. The multi-function smart wristlet 100 mentioned in the claim 1 has an emergency button 700, as shown in FIG. 2. The button sends an alert signal to the authorities and family members through wireless connection when pressed.

29. The emergency button 700 mentioned in the claim 1 can be voice activated through the person's verbal commands.

30. The multi-function smart wristlet 100 mentioned in the claim 1 has a digital key 800, as shown in FIG. 2. A digital remote on wrist that can open up different locks, such as garage, house door, and car etc. It is not limited to the functions mentioned above; other similar functions may also be used with the digital keys 800.

31. The multi-function smart wristlet 100 mentioned in the claim 1 has a music player button 900, as shown in FIG. 2. It can wirelessly, through Bluetooth 1000, hook up with any Bluetooth device 1200 and play music through them. The music player button 900 can also be embedded with an electronic chip to perform the radio function. The music player button 900 can have “we chat” function that people can talk with each other using the multi-function smart wristlet.

32. All data obtained from this wristlet mentioned in the claim 1 can be uploaded onto a synced app 1100 through Bluetooth 1000.

33. The synced app 1100 mentioned in claim 1 contains recorded data that can be used for reviewing health conditions and can be accessed by authorities such as doctors, nurses, etc.

34. The multi-function smart wristlet 100 mentioned in the claim 1 is not limited to these functional buttons aforementioned. It can be extended to include other functional buttons in general as needed.

35. Individual functions of each button mentioned in the claim 1 can be coordinated to perform other functions.

36. The main body of the multi-function smart wristlet mentioned in the claim 1 can be made of latex-free Thermoplastic elastomers (TPE), Polyvinyl Chloride (PVC), Thermoplastic polyurethane (TPU), and so on. The clasp is made of stainless steel.
